(19) United States
(12) Patent Application Publication
(10) Pub. No.: US 2026/0061216 A1
(43) Pub. Date: Mar. 5, 2026

(54) Prescription-Controlled Medical Device Using Magnetic Component Identification

(71) Applicant: Azena Medical, LLC, Walnut Creek, CA (US)

(72) Inventors: Alexandre B. Di Sessa, Port Orange, FL (US); John Malvern Swope, Redding, CA (US)

(21) Appl. No.: 19/385,876

(22) Filed: Nov. 11, 2025

Related U.S. Application Data
(63) Continuation-in-part of application No. 18/345,825, filed on Jun. 30, 2023, now Pat. No. 12,504,300.

(51) Int. Cl. A61N 5/06; A61B 17/00; G16H 20/30

(52) U.S. Cl. CPC A61N 5/06; G16H 20/30; A61B 2017/00039; A61B 2017/00477; A61B 2017/00876; A61N 2005/0626; A61N 2005/0644

(57) ABSTRACT

A medical device system provides prescription-based treatments using magnetic signatures. The system comprises interchangeable components, such as batteries or treatment tips, each containing a predetermined arrangement of magnets that creates a unique magnetic signature. A hall effect sensor in the device detects the magnetic signature based on magnetic field characteristics, particularly field strength polarities corresponding to the quantity of magnets present. A controller automatically loads specific preset procedures and treatment parameters from memory based on the detected magnetic signature, without requiring user selection. Different components having different magnetic signatures enable different sets of procedures, power settings, and treatment protocols. The system includes wireless communication for receiving prescription data from healthcare providers, with the prescription data specifying which magnetic signatures are authorized for a patient and limiting the number of treatment sessions. The magnetic signature serves as a physical prescription that configures the device for prescribed treatments while preventing unauthorized use.

### CROSS-REFERENCE TO RELATED APPLICATIONS

This application is a continuation-in-part of U.S. application Ser. No. 18/345,825, filed Jun. 30 2023, which is hereby incorporated by reference herein in its entirety including all references an appendices cited therein.

### TECHNICAL FIELD

This disclosure pertains to medical devices, and more particularly, but not by way of limitation, to medical devices that use magnetic signatures to identify and authenticate different tip attachments (peripherals) and also, prescription-based treatments. The medical devices incorporate hall effect sensors that detect magnetic signatures from interchangeable components, where different quantities and arrangements of magnets encode specific treatment protocols and prescription parameters. The magnetic signature system enables automatic device configuration, prevents unauthorized use, and ensures treatment compliance with healthcare provider prescriptions.

### SUMMARY

According to some embodiments, the present disclosure is directed to a medical device system comprising a first portion comprising a predetermined arrangement of one or more magnets that creates a magnetic signature; a second portion comprising a hall effect sensor configured to distinguish between different magnetic signatures based on magnetic field characteristics of strength and polarity; and a controller configured to automatically load a specific set of preset procedure protocols from among a plurality of different sets of preset procedures based on which magnetic signature is detected when the first portion is connected to the second portion, wherein different first portions having different magnetic signatures enable different sets of preset procedures without user selection.

In some embodiments, the magnetic signature is defined by at least one of: a quantity of magnets, a magnetic field strength, a magnetic pole arrangement, or a spatial distribution of magnets. The first portion may be one of a plurality of interchangeable batteries, each battery having a different number of magnets creating a unique magnetic signature. Alternatively, the first portion may be one of a plurality of interchangeable tips, each tip having magnets arranged to identify a specific therapeutic procedure.

According to some embodiments, each tip is associated with treatment of a specific body part, tissue type or color and the magnetic signature automatically configures power settings and treatment duration for that body part, tissue type or color. The system may further comprise a wireless communication module configured to receive prescription data that defines which first portion a patient is authorized to use and for how many treatment sessions. The controller verifies that the detected magnetic signature matches an authorized prescription before enabling operation.

In some embodiments, rotation of the first portion relative to the second portion enables user navigation through parameters within the loaded set of preset procedures while maintaining the magnetic signature detection. The system may include a base hub separate from the second portion, wherein the second portion wirelessly communicates procedure information to a display on the base hub.

According to some embodiments, the controller stores at least 40 different preset procedures in memory, and the magnetic signature determines which subset of the 40 procedures becomes accessible. The magnetic signature enables both a procedure type and a power setting simultaneously upon attachment. The hall effect sensor can distinguish between at least five different magnetic signatures to enable at least five different operational modes.

Some embodiments are directed to a method of delivering prescribed medical treatments, comprising providing a medical device having a hall effect sensor and a memory storing multiple treatment protocols; attaching a prescription component to the medical device, the prescription component containing magnets arranged in a pattern that creates an identifiable magnetic signature; detecting the magnetic signature via the hall effect sensor; automatically loading only those treatment protocols from the memory that correspond to the detected magnetic signature, without requiring user selection of protocols; and restricting device operation to the loaded treatment protocols.

The method may further comprise receiving wireless prescription data from a healthcare provider before providing the prescription component; and selecting the prescription component having the magnetic signature that matches the prescription data. In some embodiments, the method includes counting uses of the loaded treatment protocols; and disabling the medical device after a predetermined number of uses encoded in the prescription component. Different prescription components enable different combinations of power level, treatment duration, and treatment area based solely on their magnetic signatures.

According to some embodiments, the present disclosure is directed to a prescription-based photobiomodulation system comprising a handheld device comprising a hall effect sensor and a controller; a set of interchangeable components, each component containing magnets positioned to create a distinct magnetic signature that corresponds to a specific medical prescription; wherein the controller is configured to detect which component is attached based solely on the magnetic signature sensed by the hall effect sensor, automatically configure operational parameters including power level, treatment duration, and number of allowed uses based on the detected magnetic signature, and prevent modification of the operational parameters beyond those defined by the magnetic signature.

The system may further comprise a wireless interface through which the controller receives updates that modify which operational parameters are associated with each magnetic signature. In some embodiments, the component is a battery that both powers the handheld device and defines the medical prescription through its magnetic signature. The magnetic signature may be created by varying at least one of: the number of magnets from one to ten, the relative positions of magnets, or the polarities of magnets.

### BRIEF DESCRIPTION OF DRAWINGS

Exemplary embodiments are illustrated by way of example and not limitation in the figures of the accompanying drawings, in which like references indicate similar elements.

FIG. 1 is an example environment where aspects of the present disclosure can be implemented for use.

FIG. 2 is an exploded view of the base battery portion.

FIG. 3 is a view of the male adapter and a partial cutaway of the terminal end of the base battery portion.

FIGS. 4 and 5 collectively illustrate magnetic fields interacting with a hall effect sensor.

FIG. 6 is a schematic view of another example UI on a display.

FIG. 7 is a flowchart of an example method.

FIG. 8 is a flowchart of another example method.

FIG. 9 is a simplified block diagram of a computing system, in accordance with some embodiments.

FIG. 10A shows the device battery body separated from a cover ring that secures magnets in position at the spring attachment interface.

FIG. 10B shows the cover ring attached to the device battery body with the magnets secured in position.

FIG. 10C shows an interchangeable treatment tip with threaded attachment, fiber optic element, and magnets.

FIG. 11 illustrates a schematic diagram of the system architecture.

FIG. 12 shows the device body separated from a cover ring assembly having six magnets.

FIG. 13 shows the device body separated from a cover ring assembly having four magnets.

FIG. 14 shows interchangeable treatment tips with different geometries and handle configurations.

FIG. 15A is a flowchart of the main method of delivering prescribed photobiomodulation treatments.

FIG. 15B is a flowchart of the component identification sub-method.

FIG. 15C is a flowchart of the treatment delivery sub-method.

FIG. 15D is a flowchart of the authorization renewal sub-method.

### DETAILED DESCRIPTION OF EXAMPLE EMBODIMENTS

### Overview

The present disclosure pertains to devices that are two-part assemblies, referred to in combination as a handpiece. For example, the handpiece comprises a battery portion and a tool portion. These handpieces can be used as medical devices to perform medical procedures such as ablation, cutting, vaporization, photobiomodulation and the like. The devices enable user interaction with a user interface (UI) on a display using a hall-effect sensor or capacitive touch via the display. Some embodiments include an integrated display, and this display can be a touchscreen with capacitive touch capabilities. The user can confirm UI selections with a tap gesture. The user can also use other gestures such as swipes to interact with the UI on the display. In other embodiments, the hall-effect sensor on the handpiece can be used to control an external display that is connected in a wired or wireless manner. The handpieces also include haptic feedback elements to confirm user selections.

In some embodiments, the base battery portion houses a battery and magnets, while the tool portion has a hall effect sensor, a display, a controller, gravity chip sensor and a tip that can be used to deliver a laser to a patient. The battery portion is associated with different procedures and procedure categories that are designated by battery size. When a first type of battery portion is detached and replaced with a second type of battery portion having a larger battery, a second type of procedure is enabled. A controller associated with the second portion can sense which type of battery portion has been attached and can select the type of procedure allowed by that battery portion. The association of procedure type is not limited to battery size but can be based on any difference in between battery portions. Certain features not associated with procedure type can also be loaded by different base such as power settings, laser brightness and other customized settings.

In some embodiments, the battery portion or tool portion includes interchangeable components containing varying quantities of magnets that create distinct magnetic signatures. The quantity of magnets, ranging from one to ten, determines which specific procedures and power settings are enabled when the component is attached. Each magnetic signature corresponds to a different set of preset procedures stored in the device memory. For example, a component with two magnets may enable knee treatment procedures, while a component with four magnets may enable shoulder treatment procedures. The hall effect sensor detects the magnetic field strength, which is proportional to the quantity of magnets, and the controller automatically loads only those procedures associated with the detected magnetic signature.

Some embodiments include interchangeable tips that contain magnets arranged to create prescription-specific magnetic signatures. These tips serve as physical prescriptions that healthcare providers can provide to patients for specific treatments. When a prescribed tip is attached to the device, the hall effect sensor identifies the tip based on its magnetic signature and automatically configures the device with the appropriate treatment parameters, including power level, treatment duration, and wavelength. Each tip may include a usage counter that limits the number of treatments to a prescribed amount, after which the tip becomes disabled, requiring the patient to return to their healthcare provider for follow-up evaluation.

In some embodiments, the devices include wireless communication capabilities for receiving prescription data from healthcare provider systems. A healthcare provider creates a prescription specifying treatment parameters and transmits it to the device via a cloud-based server. The prescription data includes authentication information that must match the magnetic signature of the attached component before the device enables operation. This dual authentication system ensures that patients can only use components that have been specifically prescribed for them, preventing unauthorized use and ensuring treatment compliance.

Some embodiments support outcome tracking and prescription optimization through a feedback loop system. Patients can capture photographs or videos with their mobile phones or other outcome data after treatments, which are transmitted to the healthcare provider for evaluation via the custom app that connects to the medical device. An artificial intelligence system may analyze the outcome data to assess healing progression and recommend prescription modifications. Based on this analysis, healthcare providers can remotely update prescription parameters, which are transmitted to the device for implementation in subsequent treatment sessions. This creates a continuous optimization cycle where treatment parameters are adjusted based on actual patient outcomes.

### EXAMPLE EMBODIMENTS

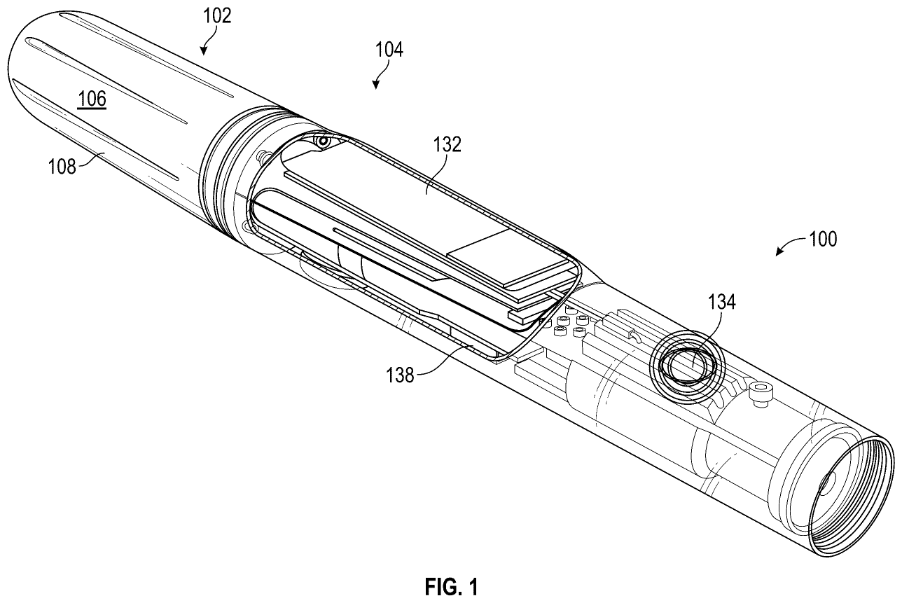

Referring now to FIG. 1, which illustrates an example architecture where aspects of the present disclosure are implemented. The example device 100 is a hand-held unit that is comprised of two pieces, a battery portion 102 (may be referred to as a first portion) and a tool portion 104 (may be referred to as a second portion). In some embodiments, the battery portion 102 includes an electrical energy storage module, such as a battery 106 enclosed in a housing 108.

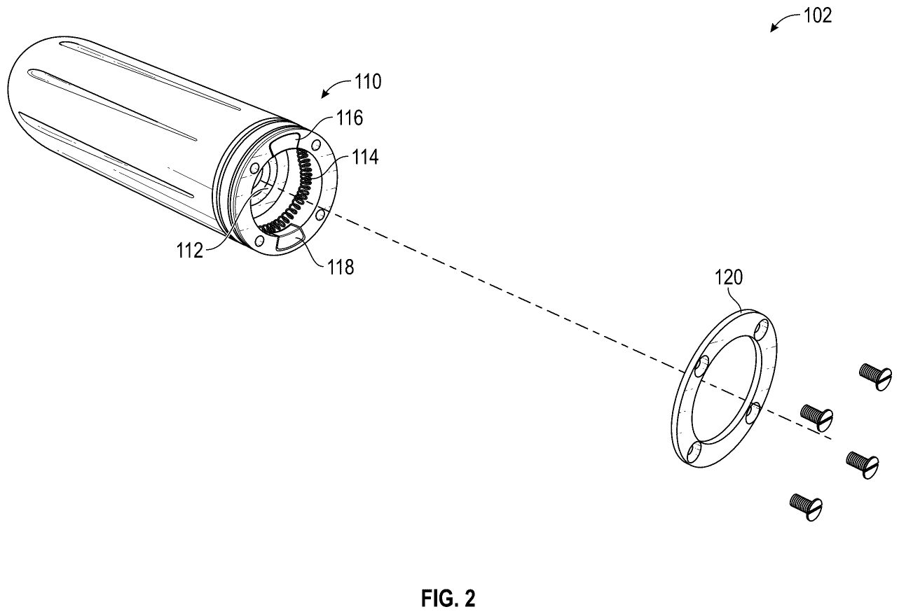

Referring now to FIGS. 1-3 collectively, the battery portion 102 includes a terminal end 110 having a first conductor 112, a spring-loaded resilient element 114, a first magnet 116, a second magnet 118, and a spacer 120. The terminal end 110 has a collar 122 that extends out and forms a female receiver that mates with a male adapter of the tool portion 104, as will be discussed in greater detail herein.

The first magnet 116 and the second magnet 118 are located on the terminal end of the collar 122 and are spaced at a 180-degree interval relative to one another, although other spacings can be used. The spacer 120 is configured to cover and lock the first magnet 116 and the second magnet 118.

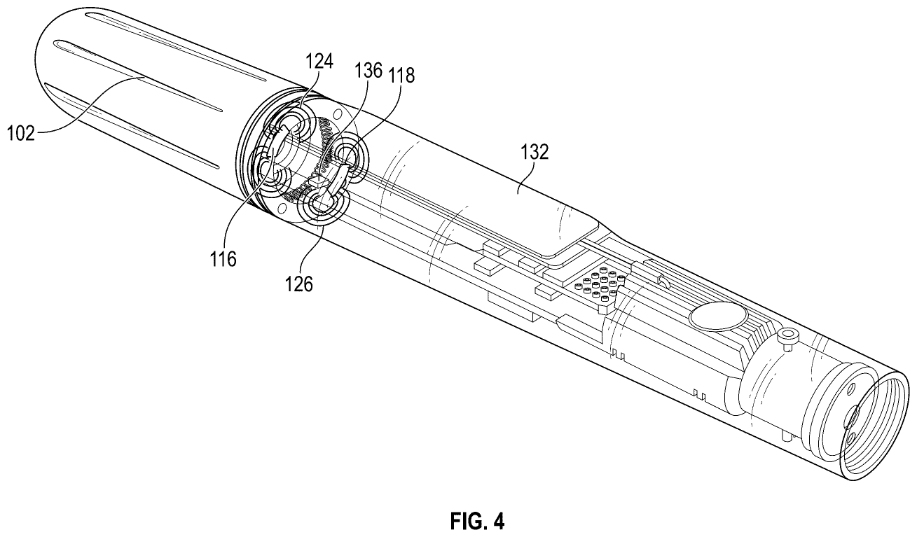

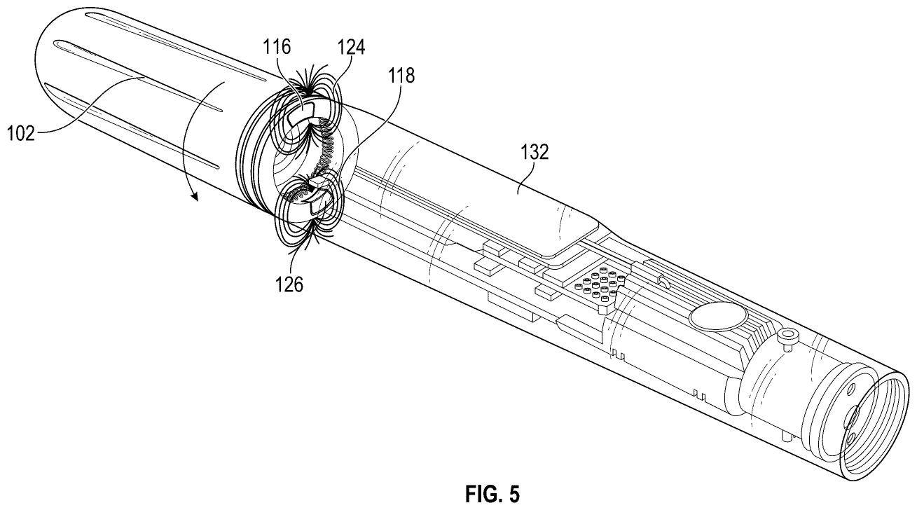

The first magnet 116 produces a first magnetic field (north pole) 124 and the second magnet 118 produces a second magnetic field (south pole) 126 (see FIGS. 4 and 5). These magnetic fields are distinguishable from one another by a hall effect sensor integrated into the tool portion 104.

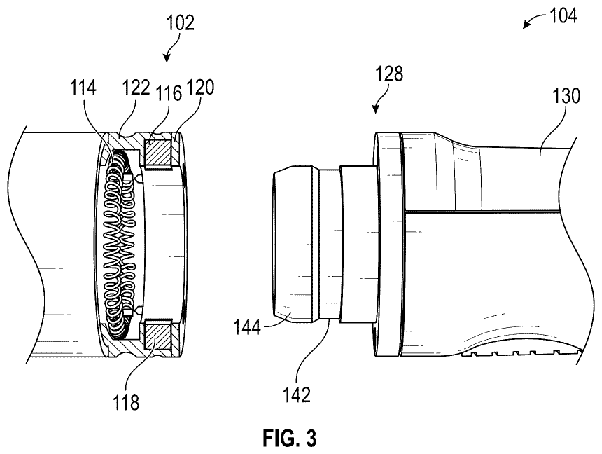

The tool portion 104 has a male adapter 128, a housing 130, a display 132, a haptic element 134, a hall-effect sensor 136, and a controller 138. The male adapter 128 is best illustrated in FIG. 3. The male adapter 128 acts a second conductor that mates with the first conductor 112 when the battery portion 102 is joined to the tool portion 104. When the battery portion 102 is joined to the tool portion 104, components of the tool portion 104 can receive energy from the battery 106 such as the display 132, the controller 138, and the haptic element 134. It is noteworthy that the haptic element 134 can be located anywhere inside the tool portion 104.

In FIG. 3, the male adapter 128 is a tubular extension that includes a circumferential groove collar 142 and a tapered pre-loaded angle end 144. When the battery portion 102 and the tool portion 104 are being joined, the tapered end 144 is inserted into the resilient spring element 114 and the user continues to push the battery portion 102 and the tool portion 104 together until the resilient element 114 seats into the circumferential collar 142. This action provides a releasable locking force that keeps the battery portion 102 and the tool portion 104 together until separated by the user. The battery portion 102 and the tool portion 104 are shown in association FIG. 3. While in some embodiments, the resilient element 114 is a spring, the resilient element 114 could also be an O-ring, gasket, or the like.

Referring to FIGS. 1-5, the controller 138 comprises a processor and memory. The memory can store instructions that can be executed by the processor to perform various functions. In one example, the controller 138 can detect when a battery portion is attached. Again, the battery portion powers the components of the tool portion with energy from a battery in the battery portion. As noted above, each battery portion can be associated with a procedure type. For example, a first battery portion can enable an ablation procedure, whereas a second battery portion can enable a cutting procedure. In some instances, this is tied to the power level of the battery of the battery portion, but this is not intended to be limiting. In some examples, the power level of two distinct battery portions is equal, yet they are associated with two different procedures. The user may distinguish these two battery portions by having housings with two different geometrical configurations that have a distinct look and feel to the user. Of note, the battery portion can also act as a custom prescription of use to the device. As an example, the product can be used in consumer applications and such embodiments may have a custom base that will have pre-loaded procedures which will be referred to as a “prescription”. Once attached, it will enable the user to utilize the procedures as prescribed by a doctor. In some instances, procedures, prescriptions, or instructions can be transmitted over a network and received by the device 100. For example, instructions can be transmitted from a doctor's office to the device in the patient's home.

Once the procedure type has been enabled, the controller 138 can control the display 132 and allow a user to make selections using input from the user rotation the battery portion 102. Rotation of the battery portion 102 causes the two magnets to interact with the hall effect sensor 136. Rotation of the battery portion 102 in a first direction causes the display to scroll in a first direction, whereas rotation of the battery portion 102 in a second direction causes the display 132 to scroll in a second direction. In one embodiment, the first direction is forward and the second direction is backward. In more detail, when the first magnet 116 is near the hall effect sensor 136, the first magnetic field 124 is sensed by the hall effect sensor 136; this is interpreted by the controller 138 as a forward scroll. When the second magnet 118 is near the hall effect sensor 136, the second magnetic field 126 is sensed by the hall effect sensor 136; this is interpreted by the controller 138 as a backwards scroll. This relates to the polarity of the magnets, where one magnet has a first polarity, and the second magnet has an opposing, second polarity

Thus, in response to the signals received by the hall effect sensor and magnets 136, the controller 138 can cause the display 132 to change. In sum, the battery portion and the tool portion are joined together in such a way that rotation of the battery portion relative to the hall effect sensor to interact with one another in such a way that a user can control the display. To be sure, some embodiments can include only one magnet that can be used to interact with the UI and display 132. Other sensors can also be used such as proximity sensors and gravity sensors. In this way, unidirectional scrolling is enabled. At a minimum this allows the controller 138 to move a cursor of the display in response to the direction of rotation.

An example UI change caused by rotation of the battery portion is shown FIGS. 4 and 5 collectively. In this example, rotation of the battery portion 102 in a first direction causes the controller to present Procedure B and continued rotation of the battery portion 102 in the first direction causes the controller to present Procedure C. The user can rotate the battery portion 102 in a second direction to scroll back to Procedure B or the user can continue rotating the battery portion 102 in the first direction until Procedure B is in view.

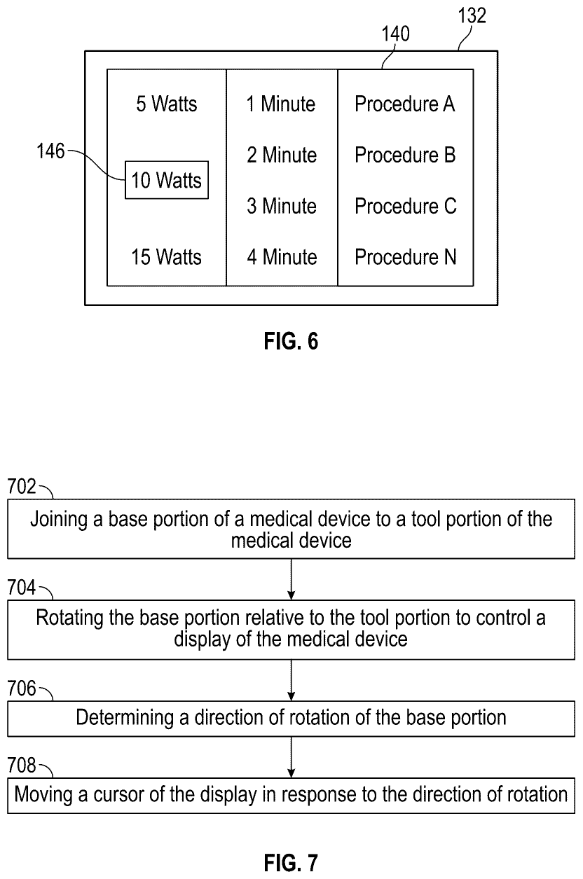

Another example is illustrated in FIG. 6, which illustrates a UI 140 that is presented on the display 132. The UI 140 includes a cursor 146, and this cursor 146 can be moved by forward or backward rotation of the battery portion 102. Again, this includes using the hall effect sensor to detect what magnet is in proximity to the hall effect sensor, to dictate the scrolling direction. To be sure, this configuration is not intended to be limiting but is an example. In other instances, the cursor can remain fixed, while UI elements are scrolled through and highlighted inside the cursor.

Referring to FIGS. 1-6, regardless of how the UI element is highlighted, when the user desires to select what is being presented on the display 132, the user can tap the display 132. This is due to the fact that in some instances, the display 132 is a touchscreen that enables gestures. It is an advantage that the user can scroll through a UI on the display 132 by rotating the battery portion 102, because this limits user touches of the display 132, which may contaminate the display 132. A selection can be made by pushing the button located at the end of the base as well. In some embodiments, the handpiece can integrate a microphone that can receive voice commands that allow a user to select a procedure rather than touch the display. However, in some embodiments, the controller 138 can enable any number of gestures so that the user can utilize the display 132 to make selections or scroll through menus. Once the selection is made, the controller 138 may activate the haptic element 134 is activated to vibrate the handpiece to confirm the selection.

The controller 138 can then allow the user to perform a medical procedure in accordance with the procedure enabled by the battery portion and the selection(s) made by the user through rotation of the battery portion. The controller 138 can be used to control laser production by a laser source (not shown) integrated into the tool portion, and laser emission through the end of the device.

FIG. 7 is a flowchart of an example method of the present disclosure. The method includes a step 702 of joining a battery portion of a medical device to a tool portion of the medical device. To be sure, the battery portion comprises one or more magnets and the tool portion comprises a hall effect sensor. Next, the method includes a step 704 of rotating the battery portion relative to the tool portion to control a display of the medical device.

The method can include a step 706 of determining a direction of rotation of the battery portion. This is accomplished when a hall effect sensor detects either the presence of a first magnet or a second magnet (and in some embodiments, the difference in polarity of the magnets). Each of these magnets produces a unique magnetic field that allows the hall effect sensor to differentiate which magnet is in proximity to the hall effect sensor. For example, when the first magnet is in proximity, the battery portion has been rotated forward. When the second magnet is in proximity, the battery portion has been rotated backward.

Next, the method includes a step 708 of moving a cursor of the display in response to the direction of rotation. This can alternatively include scrolling through programs or other types of options presented on the UI.

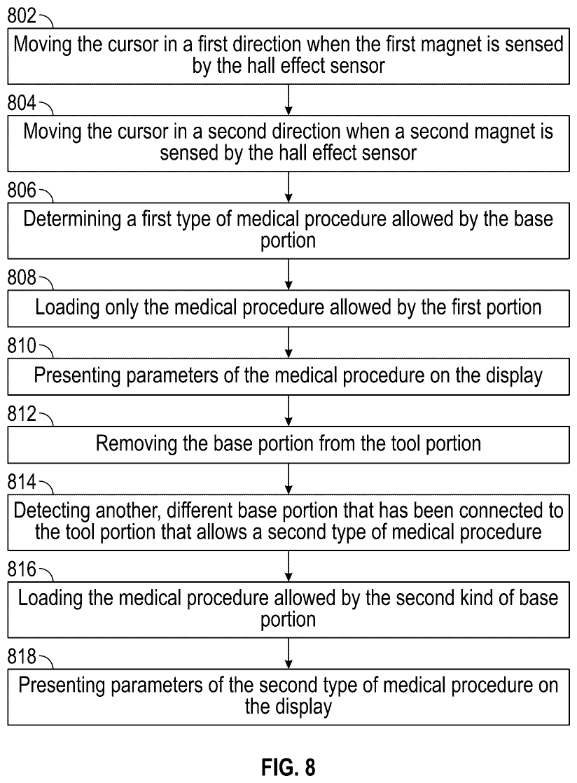

FIG. 8 is a flowchart of a related method of the present disclosure. The method can include a step 802 of moving the cursor in a first direction when the first magnet is sensed by the hall effect sensor and a step 804 of moving the cursor in a second direction when a second magnet is sensed by the hall effect sensor.

The method can also include a step 806 of determining a first type of medical procedure allowed by the battery portion and a step 808 of loading only the medical procedure allowed by the first portion. Next, the method includes a step 810 of presenting the parameters of the medical procedure on the display. This allows the user to fine-tune the parameters of the procedure. In some instances when a prescription base is attached, no selections need to be made. The procedures can be loaded automatically and the user can only select to start each of the prescribed procedures.

The method includes a step 812 of removing the battery portion from the tool portion. The method then includes a step 814 of detecting another, different battery portion that has been connected to the tool portion that allows a second type of medical procedure. Next, the method includes a step 816 of loading the medical procedure allowed by the second kind of battery portion, and a step 818 of presenting parameters of the second type of medical procedure on the display.

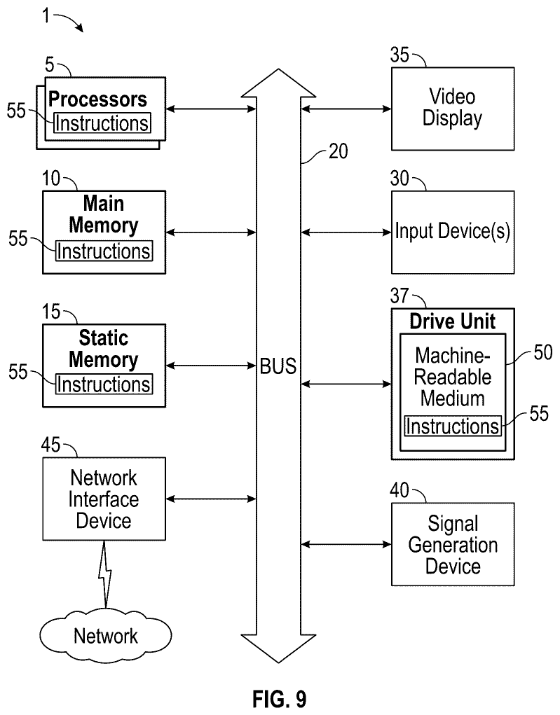

FIG. 9 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or decentralized) network environment. The machine may be a personal computer (PC), a tablet PC, a set-top box (STB), a personal digital assistant (PDA), a cellular telephone, a portable music player (e.g., a portable hard drive audio device such as a Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. When enabled Wi-Fi connectivity with cloud servers, new procedures and prescriptions can be loaded on-demand via a cloud, as noted above.

The computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a BLE encrypted network or via the network interface device 45 utilizing any one of a number of well-known transfer protocols (e.g., Hyper Text Transfer Protocol (HTTP or Incognito). While the machine-readable medium 50 is shown in an example embodiment to be a single medium, the term “computer-readable medium” should be taken to include a single medium or multiple media (e.g., a centralized or decentralized database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

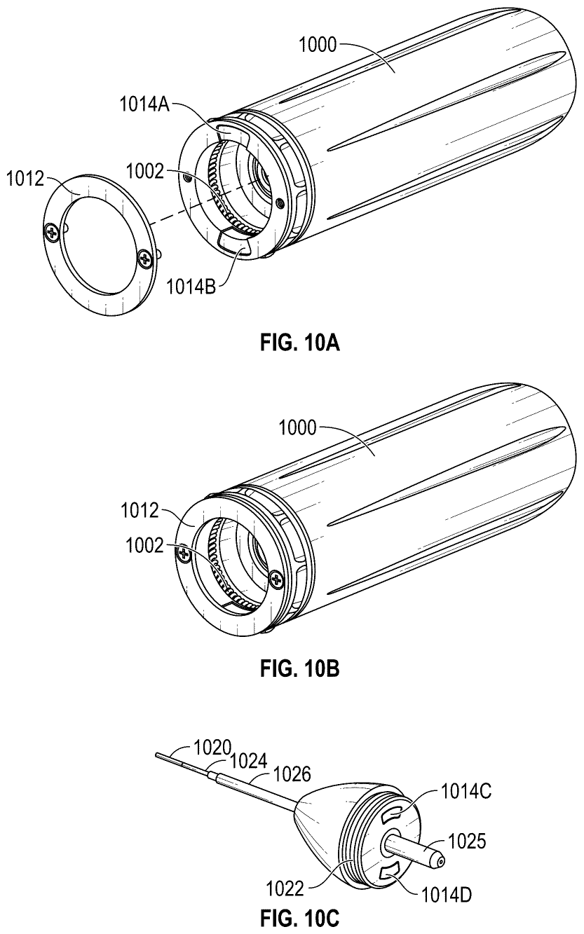

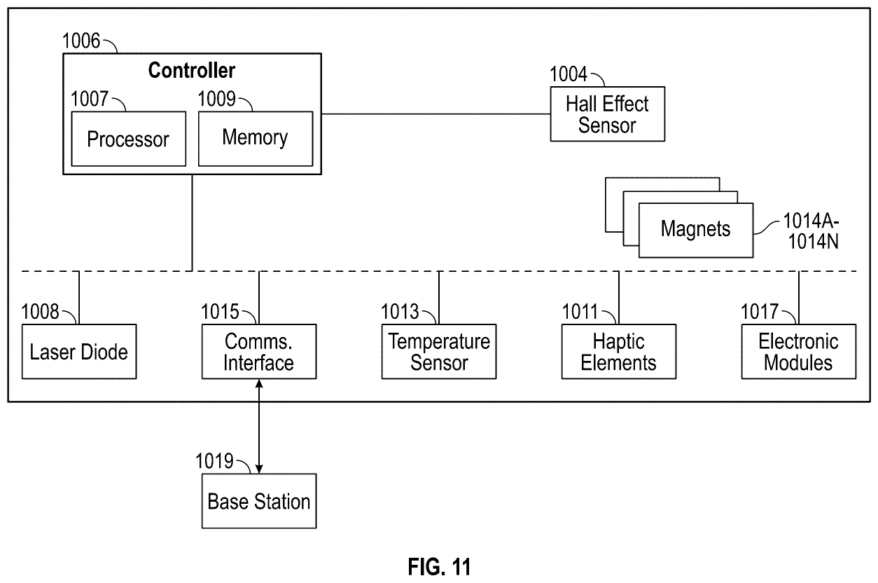

FIGS. 10A-10C show an example device of the present disclosure. The following descriptions will also reference the circuit diagram of FIG. 11. FIG. 10A illustrates a photobiomodulation device in a disassembled state. A battery body 1000 has a substantially cylindrical housing with a spring attachment interface 1002 disposed at a forward end. The spring attachment interface 1002 includes mechanical features configured to receive interchangeable components. A cover ring 1012 is shown separated from the battery body 1000. The cover ring 1012 comprises an annular structure dimensioned to mechanically engage the spring attachment interface 1002 and secure magnets in position.

Two magnets 1014A and 1014B are discrete components that create the magnetic signature detected by the hall effect sensor. The quantity, arrangement, positioning and strength of magnets 1014A and 1014B determine the specific magnetic field characteristics. The magnets 1014A and 1014B are housed within a silver ring component that positions the magnets to create a specific magnetic pull. The magnetic pull refers to the magnetic field strength that the hall effect sensor detects. The magnetic field must be precisely tuned to the hall effect sensor; if the magnetic field varies even slightly from the expected characteristics, detection fails and the controller does not recognize the component.

The spring attachment interface 1002 disposed at the forward end of the device body 1000 comprises a spring-based attachment mechanism configured to receive and secure interchangeable components. The spring attachment interface 1002 provides resilient mechanical engagement that holds attached components in position while allowing for deliberate turning adjustment, removal and replacement. The spring mechanism applies retention force to maintain secure attachment during use while permitting component interchange when desired by the user.

The spring attachment interface 1002 includes a receptacle geometry dimensioned to accept the cover ring 1012 and position the magnets 1014A and 1014B at a precise distance from the hall effect sensor 1004. The spring mechanism ensures consistent positioning of magnets relative to the hall effect sensor 1004 by maintaining constant contact pressure between the attached component and the device body 1000. The spring attachment interface 1002 may include alignment features such as grooves, ridges, or positioning surfaces that ensure proper rotational and axial alignment of attached components. The alignment features ensure that magnets within attached components are positioned within the detection range of the hall effect sensor 1004.

The spring attachment interface 1002 is configured to receive multiple types of interchangeable components including magnetic ring assemblies (such as cover ring 1012 with magnets 1014A and 1014B) and treatment tips (such as treatment tip 1020). The spring mechanism maintains consistent positioning of magnets relative to the hall effect sensor 1004 regardless of which type of component is attached, enabling reliable magnetic signature detection across different component types.

In embodiments where treatment tips attach directly to the device body 1000, the spring attachment interface 1002 additionally provides alignment between the laser diode 1008 and the fiber optic element of the treatment tip, ensuring efficient optical coupling for laser energy transmission.

Having described the external mechanical attachment system, the internal components of the battery body 1000 will now be explained. The battery body 1000 houses a hall effect sensor 1004 positioned adjacent to the spring attachment interface 1002. The hall effect sensor 1004 is configured to detect magnetic field characteristics including field strength and spatial distribution. The hall effect sensor 1004 also functions as a gyroscopic sensor configured to sense direction and movement for motion-based procedure controls. A controller 1006 is disposed within the battery body 1000 and is operatively connected to the hall effect sensor 1004. A laser diode 1008 is disposed within the battery body 1000 and is configured to emit coherent laser light at controlled wavelengths suitable for photobiomodulation therapy, rather than LED-based light. The laser diode 1008 is coupled to optical components including lenses configured to deliver energy to tissue in a controlled manner. A wireless communication module 1009 is disposed within the battery body 1000 and is configured to communicate with external devices. The magnetic signature system provides cost advantages compared to RF chip implementations and security advantages against unauthorized access attempts.

The magnetic signature system provides significant advantages over alternative identification technologies such as RF chips or electronic identification systems. The cost of implementing the magnetic signature system is substantially lower than RF-based alternatives, as the variable component is the passive magnets rather than active electronic components. The hall effect sensor remains fixed in the device body while only the magnets change between different components, making the system more upgradable through mechanical changes rather than electronic modifications. The magnetic signature system provides enhanced security compared to RF-based systems, as magnets must be very precisely tuned to specific magnetic field characteristics. If the magnetic field varies even slightly from the expected signature, detection fails and the controller does not recognize the component. This precise tuning requirement makes unauthorized duplication significantly more difficult compared to RF systems which may be more susceptible to electronic cloning or hacking attempts. When the controller determines that an attached component is not authorized or that the authorized usage count has been exhausted, the device becomes completely inoperable, functioning as a non-responsive unit until proper authorization is restored. This ensures treatment compliance and prevents unauthorized use of the device outside prescribed parameters.

FIG. 10B shows the cover ring 1012 engaged with the spring attachment interface 1002 of the battery body 1000, with the magnets 1014A and 1014B secured in position between the cover ring 1012 and the battery body 1000. In this assembled configuration, the magnets 1014A and 1014B are positioned proximate to the hall effect sensor 1004. The hall effect sensor 1004 detects the magnetic pull generated by the magnets 1014A and 1014B and provides output signals to the controller 1006. The controller 1006 analyzes the magnetic field characteristics to identify a magnetic signature corresponding to the specific quantity and arrangement of the magnets 1014A and 1014B. The controller 1006 compares the detected magnetic signature to stored signature data in memory. Each stored signature is associated with a specific subset of treatment procedures. Upon identifying a match, the controller 1006 automatically loads only those procedures corresponding to the detected magnetic signature, thereby restricting available device functions. The controller simply enables the appropriate subset from approximately 40 different preset procedures stored in memory.

FIG. 10C illustrates an interchangeable treatment tip 1020 configured for optical coupling with the battery body 1000. The treatment tip 1020 comprises a tip body having a threaded battery portion 1022 configured for threaded engagement with the battery body 1000 or with the spring attachment interface 1002. A fiber optic element 1024 extends through the tip body from a proximal end 1025 to a distal end 1026. The fiber optic element 1024 is configured to transmit laser energy from the laser diode 1008 to tissue. Magnets 1014C and 1014D are disposed within or on the tip body. The magnets 1014C and 1014D create a magnetic signature detectable by the hall effect sensor 1004 when the treatment tip 1020 is attached to the battery body 1000.

Different treatment tips contain different quantities of magnets, creating distinct magnetic signatures corresponding to specific treatment parameters including power level, treatment duration, and authorized usage count. When the authorized usage count is exhausted, the tip does not become permanently unusable; rather, the tips are wirelessly reloadable with updated treatment parameters after healthcare provider authorization. Treatment tips may be configured for specific medical applications including sports medicine treatments for knee injuries, shoulder injuries, and Achilles tendon injuries, as well as dental applications. Tips may also be configured for preconditioning treatments before radiotherapy to prevent tissue damage, or for treating radio dermatitis.

The device body 1000 houses the laser diode 1008, which generates coherent laser light at controlled wavelengths. When the interchangeable treatment tip 1020 is threadedly engaged with the device body 1000 via the threaded battery portion 1022, the proximal end of the fiber optic element 1024 becomes aligned with an optical output of the laser diode 1008. This alignment creates an optical coupling that allows laser energy generated by the laser diode 1008 to be transmitted into the fiber optic element 1024. The fiber optic element 1024 then transmits the laser energy through the length of the tip body to the distal end 1026, where the energy is delivered to tissue. The attachment interface 1002 includes mechanical features that ensure proper alignment between the laser diode 1008 and the fiber optic element 1024 when the treatment tip 1020 is fully engaged. The optical coupling system allows different treatment tips with different fiber optic configurations and spot sizes to be interchanged while maintaining efficient laser energy transmission from the device body 1000 to the treatment area.

The interchangeable treatment tips may be configured for specific anatomical treatments based on medical specialty. In sports medicine applications, tips may be configured for knee injury treatment, shoulder injury treatment, or Achilles tendon treatment, with each tip containing a magnetic signature that automatically loads protocols specific to that anatomical location. In dental applications, tips may be configured for specific oral procedures with appropriate power levels and treatment durations. Each tip configuration incorporates magnets arranged to create a unique magnetic signature corresponding to the intended treatment area, ensuring that when a healthcare provider prescribes a knee treatment tip, for example, the device automatically configures itself with knee-specific treatment parameters upon tip attachment without requiring manual selection by the patient or practitioner.

FIG. 11 illustrates a schematic diagram of the system architecture. The controller 1006 comprises a processor 1007 and memory 1009. The processor 1007 executes instructions stored in the memory 1009 to perform magnetic signature detection, procedure loading, and treatment control functions. The hall effect sensor 1004 is operatively connected to the controller 1006 and provides magnetic field measurement data and motion sensing data to the processor 1007. Magnets 1014A-1014N (such as magnets 1014A and 1014B in a magnetic ring configuration or magnets 1014C and 1014D in a disposable treatment tip) create the magnetic field that the hall effect sensor 1004 detects. The memory 1009 stores the procedure database, associations between magnetic signatures and procedure subsets, usage counters, and prescription data received via wireless communication. A haptic element 1011 is operatively connected to the controller 1006 and is configured to provide haptic feedback including vibration and light signals to confirm user selections and indicate device status. A temperature sensor 1013 is operatively connected to the controller 1006 and is configured to measure tissue temperature during treatment. A communication interface 1015 is operatively connected to the controller 1006 and is configured to establish Bluetooth wireless connections with external devices. Electronic modules 1017 comprise circuitry for power management, signal processing, and device control functions.

In embodiments where a separate base station 1019 is utilized (shown schematically in FIG. 11), the Bluetooth communication interface 1015 establishes wireless communication between the device body 1000 and the base station 1019. The base station 1019 comprises a display for presenting procedure information, power settings, and treatment parameters. An activation pedal may also communicate with the device body 1000 via the Bluetooth communication interface 1015. The device body 1000, base station 1019, and activation pedal form a three-way Bluetooth communication network. Settings and procedures are adjusted by the user viewing information on the display of the base station 1019, which is separate from and external to the handheld device body 1000.

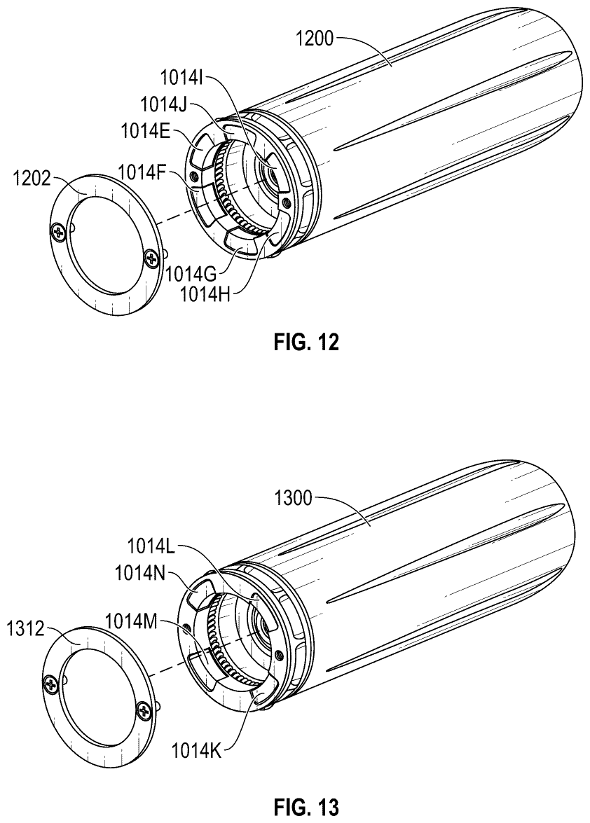

FIG. 12 illustrates the battery body 1200 separated from an alternative magnetic ring assembly comprising a cover ring 1202 and magnets 1014E-1014J. The cover ring 1202 has substantially the same external dimensions and mechanical interface features as the cover ring 1012 shown in FIG. 10A. However, the magnets 1014E-1014J differ in quantity from the magnets 1014A and 1014B shown in FIG. 10A. The different quantity of magnets 1014E-1014J creates a magnetic field with different strength characteristics, producing a magnetic signature distinguishable from that created by the two-magnet configuration of FIG. 10A. When the cover ring 1202 with magnets 1014E-1014J is attached to the spring attachment interface 1002, the hall effect sensor 1004 detects this alternative magnetic signature. The controller 1006 accesses a different entry in the stored signature library and loads a different subset of treatment procedures corresponding to the six-magnet configuration. The different procedures may include different power settings, treatment durations, or therapeutic applications compared to those associated with the two-magnet configuration as in FIGS. 10A-10C. The controller 1006 accesses a different entry in the stored signature library and loads a different subset of treatment procedures corresponding to the four-magnet configuration. The different procedures may include different power settings, treatment durations, or therapeutic applications compared to those associated with the two-magnet configuration of magnets as in FIGS. 10A-10C.

FIG. 13 illustrates the device body 1300 separated from a magnetic ring assembly comprising a cover ring 1312 and magnets 1014K-1014N. The device body 1300 has a substantially cylindrical housing with an attachment interface at a forward end, similar to the device body 1000 shown in FIGS. 10A-10B. The cover ring 1312 has an annular structure configured to mechanically engage the attachment interface and secure the magnets in position. The magnets 1014K-1014N are discrete components arranged within or secured by the cover ring 1312. The quantity of magnets shown in FIG. 13 differs from the two-magnet configuration of FIGS. 10A-10B and the six-magnet configuration of FIG. 12, creating a distinct magnetic signature. When the cover ring 1312 with magnets 1014K-1014N is attached to the device body 1300, the hall effect sensor within the device body 1300 detects this specific magnetic signature. The controller analyzes the magnetic field characteristics and loads a corresponding subset of treatment procedures associated with this particular magnet configuration. The different magnetic signature enables different operational parameters including power settings, treatment protocols, and authorized procedures compared to other magnet configurations.

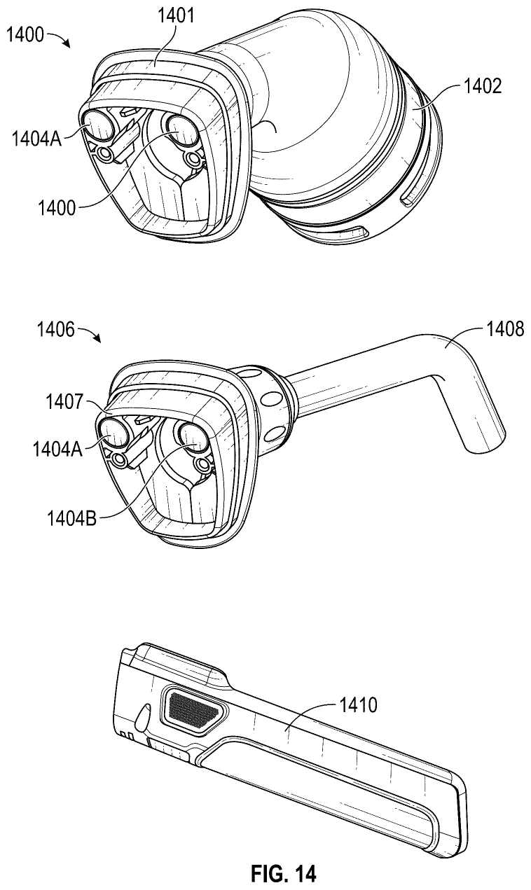

FIG. 14 illustrates interchangeable treatment tips with different geometries and magnet configurations. A first treatment tip 1400 comprises a tip body with a bulbous or enlarged housing geometry. An attachment interface 1401 is disposed at a proximal end of the first treatment tip 1400 and is configured for engagement with the battery body 1410. The first treatment tip 1400 includes a distal end 1402 configured to emit laser energy to tissue. An optical pathway extends through the tip body from the attachment interface 1401 to the distal end 1402. The optical pathway is configured to transmit laser energy to tissue. Magnets 1404A and 1404B are disposed within or on the attachment interface 1401 of the first treatment tip 1400. The enlarged geometry of the first treatment tip 1400 provides a larger spot size for treating broader anatomical areas.

A second treatment tip comprises a tip body 1406 with a handle structure extending laterally therefrom. An attachment interface 1407 is disposed at a proximal end of the tip body 1406 and is configured for engagement with the battery body 1410. The attachment interface 1407 has substantially the same configuration as the attachment interface 1401 of the first treatment tip 1400. The second treatment tip includes a distal end 1408 configured to emit laser energy to tissue. An optical pathway extends through the tip body 1406 from the attachment interface 1407 to the distal end 1408. Magnets 1404A and 1404B are disposed within or on the attachment interface 1407, creating a magnetic signature. In alternative embodiments, the second treatment tip may include a different quantity or arrangement of magnets to create a magnetic signature distinct from that of the first treatment tip 1400. The handle structure of the tip body 1406 provides a grip surface for manual manipulation during treatment. The handle structure is positioned at an angle relative to a longitudinal axis of the tip body 1406 to facilitate treatment of anatomical areas requiring angular approach or extended treatment duration requiring ergonomic support.

FIG. 14 also illustrates a battery body 1410 having a form factor optimized for handheld operation. The battery body 1410 houses internal components including a hall effect sensor, controller, laser diode, wireless communication module, haptic element, temperature sensor, and Bluetooth communication interface similar to those described in FIG. 11. The battery body 1410 is configured to receive interchangeable treatment tips via attachment at the attachment interfaces 1401 and 1407. When a treatment tip is attached to the battery body 1410, the hall effect sensor detects the magnetic signature created by the magnets within the attachment interface of the tip. The controller analyzes the detected magnetic signature and loads a corresponding subset of treatment procedures from memory. Different treatment tips containing different quantities or arrangements of magnets create distinct magnetic signatures. Each magnetic signature causes the controller in battery body 1410 to load different treatment procedures, enforce different power limits, and track usage for prescription compliance.

The system enables supervised photobiomodulation treatments wherein professional-level controlled laser delivery is administered in at-home environments under healthcare provider supervision. After authorized treatment cycles are completed, the patient captures photographs of the treatment area using a smartphone or integrated camera. The photographs are uploaded to a healthcare provider system along with treatment history data including timestamps and power levels used during each treatment. An artificial intelligence system analyzes the uploaded photographs by comparing current images to baseline images from treatment initiation. The AI system detects changes in tissue characteristics including coloration, swelling, and wound closure.

The AI system quantifies healing progression using objective metrics based on an image picture database and generates a healing assessment report. The AI-generated report provides educational value for physicians by illustrating expected healing progressions and outcomes. Over time, the system accumulates treatment data from thousands of procedures, creating a database that identifies optimal treatment protocols. For example, the database may demonstrate that six laser procedures produce superior healing outcomes compared to five procedures for a particular injury type. Upon reviewing the AI analysis and photographs, the healthcare provider transmits a wireless authorization message to the device. If the healthcare provider does not approve continuation, the device becomes inoperable until reauthorization occurs. If approved, the controller updates the authorized usage count in memory to enable additional treatment cycles, and the device automatically loads the updated prescription parameters.

The prescription renewal process is designed to minimize patient burden while maintaining healthcare provider oversight. When authorized treatment cycles are completed, the patient does not need to leave their home or visit a healthcare facility to request additional treatments. Instead, the patient initiates a prescription renewal request through a mobile application or web interface associated with the device. This request is transmitted to the healthcare provider system along with any outcome data such as photographs or sensor measurements. The healthcare provider reviews the request and outcome data, and if additional treatment is deemed appropriate, the provider approves the request through their system interface. Upon approval, the prescription data is transmitted wirelessly to the device via the wireless communication module. The controller receives the updated prescription data and automatically loads the new authorized usage count into memory without requiring any manual intervention by the patient. The device immediately becomes operational for the newly authorized treatment cycles. This automatic loading mechanism ensures seamless treatment continuation while maintaining the healthcare provider's control over treatment progression. If the healthcare provider does not approve the renewal request, the device remains inoperable until authorization is granted, functioning as a non-responsive unit that prevents unauthorized treatment.

The interchangeable, disposable treatment tips shown in FIGS. 10C and 14 enable automatic configuration of the device body for different therapeutic applications based solely on passive magnetic signatures. Each tip contains a unique magnet arrangement that, upon attachment, causes the controller to load specific procedures, enforce specific power limits, and track usage for prescription compliance. The system enables prescription-based delivery wherein healthcare providers distribute specific tips to patients, with each tip magnetically encoded to permit only authorized treatment protocols.

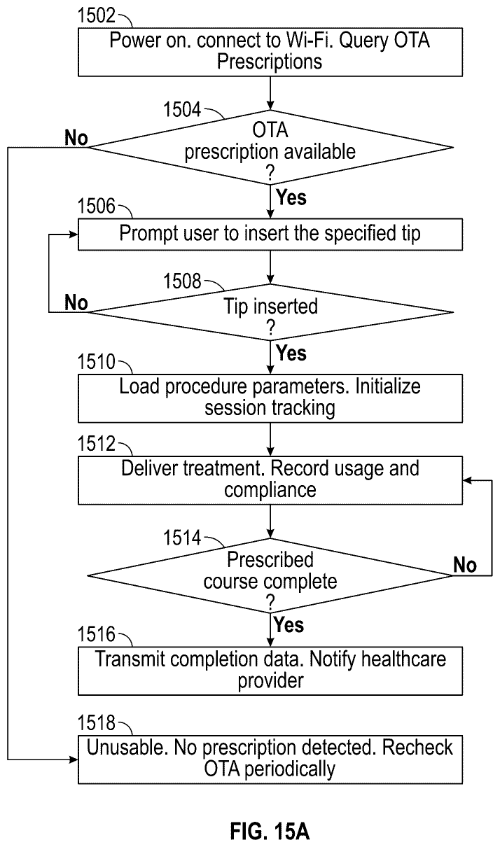

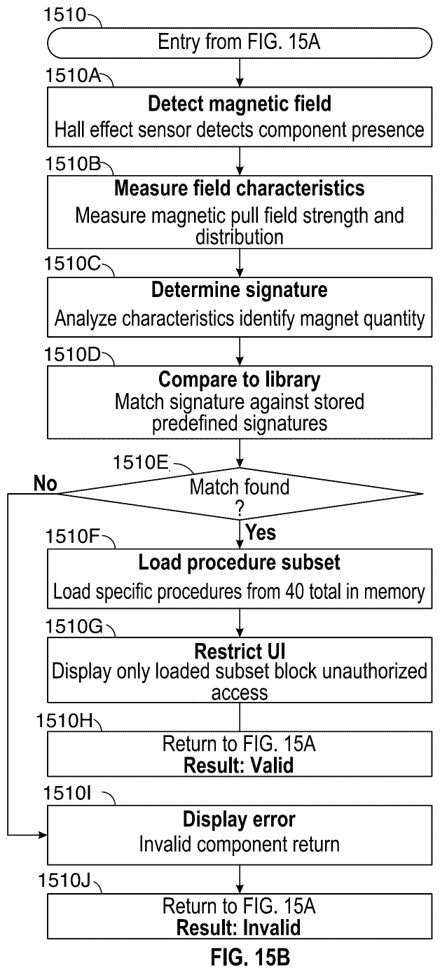

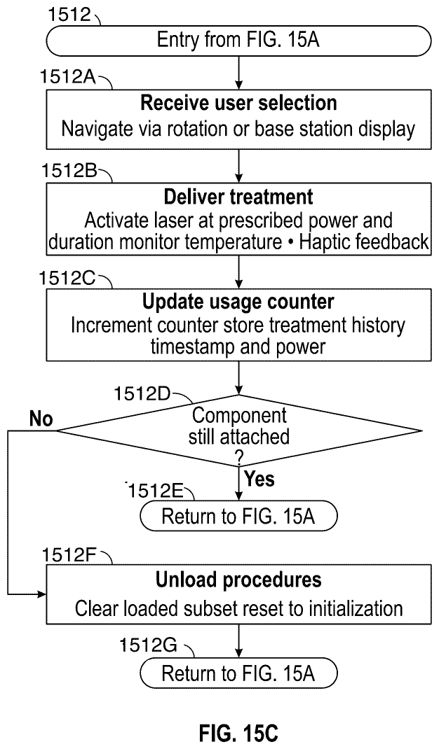

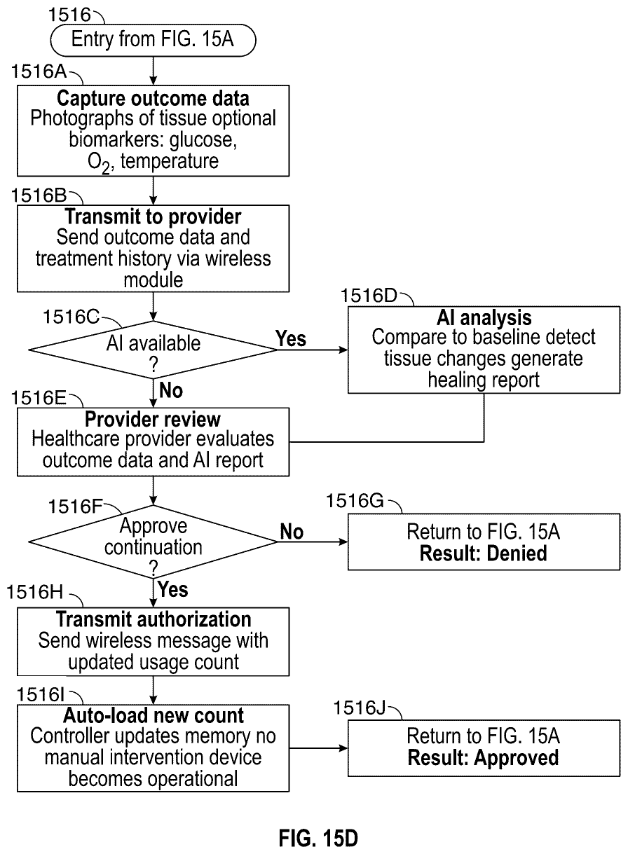

FIG. 15A illustrates a flowchart of a method for delivering prescribed photobiomodulation treatments. The method begins at step 1502 by powering on the device and establishing a Wi-Fi connection to query an over-the-air service for any available prescriptions. At decision step 1504, the controller determines whether a valid new or previously issued prescription is available. If no prescription is available, the unit is unusable and remains in a wait state, periodically rechecking for OTA prescriptions. If a prescription is available, the method advances to step 1506 where the controller prompts the user to insert the specified interchangeable tip. At decision step 1508, the controller determines whether the tip has been inserted. If the tip has not been inserted, the method continues to wait for tip insertion. Upon tip insertion, the method proceeds to step 1510 where the device verifies proper component attachment, loads the corresponding procedure parameters into active memory, and initializes session tracking, as shown in FIG. 15B. At step 1512, the device delivers the treatment and records usage and compliance data, as shown in FIG. 15C. At decision step 1514, the controller determines whether the prescribed course is complete. If additional treatment cycles remain, the method returns to step 1512. If the course is complete, the method proceeds to step 1516 to transmit completion data and notify the healthcare provider, as shown in FIG. 15D. At step 1518, the process completes. If the interchangeable tip is removed at any point during treatment, the controller detects removal and returns to step 1506 for reidentification and procedure reload.

FIG. 15B illustrates the component identification and procedure loading sub-method called from step 1510 of FIG. 15A after the user has inserted an interchangeable tip. The sub-method begins at step 1510A where the hall effect sensor detects presence of a magnetic field when an interchangeable component containing magnets is brought into proximity with the device body. The spring attachment interface mechanically secures the component while maintaining proper alignment between the magnets and the hall effect sensor. At step 1510B, the hall effect sensor measures magnetic field characteristics comprising field strength and spatial distribution of the magnetic field. The magnetic field strength, also referred to as magnetic pull, is proportional to the quantity of magnets in the component, with quantities ranging from one to ten magnets creating distinguishable signatures.

At step 1510C, the controller analyzes the measured magnetic field characteristics to determine a magnetic signature of the interchangeable component. This analysis includes evaluating the magnetic pull intensity and comparing it against expected field strength values stored in memory. At step 1510D, the controller compares the determined magnetic signature against a stored library of predefined magnetic signatures maintained in memory. Each predefined magnetic signature in the stored library is associated with a specific subset of treatment procedures, power level parameters, and authorized usage count. The comparison process identifies which entry in the signature library matches the detected magnetic signature. At decision step 1510E, if no matching signature is found in the stored library, the sub-method proceeds to step 1510I where the device displays an error message indicating invalid or unauthorized component, then proceeds to step 1510J to return to FIG. 15A with an invalid result.

If at decision step 1510E a matching signature is found, the sub-method proceeds to step 1510F where the controller automatically loads only the specific subset of treatment procedures corresponding to the detected magnetic signature from the procedure database. This automatic loading mechanism implements the concept that all procedures are pre-stored in the device, and the magnetic signature simply enables the appropriate subset. Treatment procedures not included in the specific subset remain inaccessible and cannot be selected or viewed by the user. At step 1510G, the controller restricts the user interface to display only the loaded specific subset of treatment procedures, preventing navigation to unauthorized procedures. The sub-method then proceeds to step 1510H to return to FIG. 15A with a valid result.

FIG. 15C illustrates the treatment delivery and usage tracking sub-method called from step 1512 of FIG. 15A. The sub-method begins at step 1512A where the device receives user selection of a treatment procedure from the loaded specific subset of procedures. User navigation through available procedures may occur through rotation of the interchangeable component relative to the device body, with the hall effect sensor detecting directional rotation based on magnet polarity. Alternatively, if a separate base station is utilized, the user views procedure information on the base station display and makes selections via the wireless communication interface. At step 1512B, upon confirming selection through haptic feedback or display confirmation, the device delivers a treatment cycle according to the selected treatment procedure by activating the laser diode at the prescribed power level and duration. During treatment delivery, the controller monitors treatment parameters including duration, power output, and tissue temperature via the temperature sensor. Haptic feedback is provided to confirm treatment initiation and completion.

At step 1512C, upon completion of the treatment cycle, the controller increments a usage counter that tracks the total number of treatment cycles delivered with the current interchangeable component. The controller stores treatment history data in non-volatile memory including timestamp, power level used, treatment duration, and temperature measurements for each completed treatment cycle. At decision step 1512D, the controller determines whether the interchangeable component remains attached to the device body by monitoring the hall effect sensor output. If the interchangeable component remains attached, the sub-method proceeds to step 1512E and returns to the main method at decision step 1514 of FIG. 15A to evaluate whether the prescribed course is complete. If the hall effect sensor detects absence of the magnetic field indicating component removal, the sub-method proceeds to step 1512F where the controller unloads the loaded specific subset of treatment procedures, clears the restricted user interface, and resets the device to an initialization state. The sub-method then proceeds to step 1512G and returns to the main method at step 1508 of FIG. 15A to await component reattachment.

FIG. 15D illustrates the outcome capture and authorization renewal sub-method called from step 1516 of FIG. 15A. The sub-method begins at step 1516A where the device prompts the patient to capture outcome data representing treatment results after completing the authorized usage count. The outcome data comprises photographic images of the treated tissue captured using a smartphone or integrated camera. The photographs document visible healing progression including changes in coloration, swelling, wound closure, and tissue pigmentation. The outcome data may further comprise biomarker measurements selected from blood glucose level, oxygen saturation level, and tissue temperature measured by the temperature sensor integrated into the device. At step 1516B, the wireless communication module transmits the outcome data via wireless connection to a healthcare provider system along with treatment history data including timestamps, power levels used during each treatment cycle, and temperature measurements.

At decision step 1516C, the sub-method evaluates whether artificial intelligence analysis capabilities are available at the healthcare provider system. If AI capabilities are available, the method proceeds to step 1516D where an artificial intelligence system analyzes the photographic images by comparing current images to baseline images captured at treatment initiation. The AI system detects changes in tissue characteristics including coloration, swelling, wound closure, and tissue pigmentation. The AI system quantifies healing progression using objective metrics and generates a healing assessment report comprising quantified healing metrics. The AI-generated report provides educational value for physicians by illustrating expected healing progressions and documenting outcomes across thousands of procedures in a database. If at decision step 1516C AI capabilities are not available, the method proceeds directly to step 1516E, bypassing the AI analysis.

At step 1516E, the healthcare provider reviews the outcome data and AI analysis if available to make a treatment continuation decision. The healthcare provider evaluates healing progression, treatment effectiveness, and patient compliance based on the transmitted data. At decision step 1516F, if the healthcare provider determines that continued treatment is not appropriate or if concerns about healing progression exist, or if no response is received from the healthcare provider within a predetermined timeout period, the sub-method proceeds to step 1516G and returns to the main method at step 1518 of FIG. 15A where the device becomes inoperable until proper authorization is restored. If at decision step 1516F the healthcare provider approves continuation of treatment, the method proceeds to step 1516H where the healthcare provider system transmits a wireless authorization message to the device body via the wireless communication module. The wireless authorization message comprises an updated authorized usage count specifying how many additional treatment cycles are permitted.

At step 1516I, the controller receives the wireless authorization message and automatically loads the updated authorized usage count into memory without requiring any manual intervention by the patient. The device immediately becomes operational for the newly authorized treatment cycles. This automatic loading mechanism ensures seamless treatment continuation while maintaining healthcare provider control over treatment progression. The controller may also receive updated prescription parameters including modified power levels or treatment durations based on healing progression observed in the outcome data. The sub-method then proceeds to step 1516J and returns to the main method at step 1512 of FIG. 15A, enabling the method to continue treatment delivery under the new authorization. The feedback loop comprising treatment delivery, outcome capture, AI-assisted analysis, healthcare provider authorization, and automatic loading creates a continuous optimization cycle where treatment parameters are adjusted based on actual patient outcomes.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

Aspects of the present technology are described above with reference to flowchart illustrations and/or block diagrams of methods, apparatus (systems) and computer program products according to embodiments of the present technology. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

In this description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

What is claimed is:

1. A photobiomodulation device, comprising:
a device body comprising a spring attachment interface, a hall effect sensor disposed adjacent to the spring attachment interface, a laser diode or LED, a controller operatively connected to the hall effect sensor, and memory storing a procedure database comprising a plurality of treatment procedures;
an interchangeable component comprising a plurality of magnets arranged to create a magnetic signature, the interchangeable component being attachable to the spring attachment interface and rotatable relative to the device body after attachment;
wherein the controller is configured to:
store associations between a plurality of predefined magnetic signatures and corresponding subsets of the plurality of treatment procedures;
detect the magnetic signature of the interchangeable component via the hall effect sensor when the interchangeable component is attached to the spring attachment interface;
automatically load a specific subset of treatment procedures from the procedure database based on the magnetic signature without user input;
restrict device operation to only the loaded specific subset of treatment procedures by preventing access to treatment procedures not included in the loaded specific subset; and
detect rotation of the interchangeable component relative to the device body and enable user navigation through parameters within the loaded specific subset of treatment procedures in response to the rotation.

2. The device of claim 1, wherein the magnetic signature is defined by a quantity of magnets in the plurality of magnets, the quantity being selected from 2, 4, 6, 8, 10 or more magnets.

3. The device of claim 1, wherein the interchangeable component is a battery component configured to supply electrical power to the device body, the battery component comprising a ring structure containing the plurality of magnets disposed at a terminal end of the battery component.

4. The device of claim 1, wherein the interchangeable component is a disposable treatment tip comprising a tip body having a threaded battery portion configured for threaded engagement with the spring attachment interface, a fiber optic element configured to transmit laser energy from the laser diode to tissue, and the plurality of magnets disposed within the tip body.

5. The device of claim 1, further comprising a base hub separate from the device body, the base hub comprising a display, wherein the device body further comprises a wireless communication module configured to wirelessly transmit procedure information to the base hub for display, and wherein the controller is configured to transmit updated parameter information to the base hub in response to rotation of the interchangeable component.

6. The device of claim 1, wherein the device body further comprises a haptic feedback element operatively connected to the controller, and wherein the controller is configured to activate the haptic feedback element in response to user selection of a parameter during rotation of the interchangeable component.

7. The device of claim 1, further comprising:
a first interchangeable component comprising a first plurality of magnets arranged to create a first magnetic signature; and
a second interchangeable component comprising a second plurality of magnets arranged to create a second magnetic signature distinguishable from the first magnetic signature;
wherein the controller loads a first subset of treatment procedures responsive to detecting the first magnetic signature and loads a second subset of treatment procedures different from the first subset responsive to detecting the second magnetic signature.

8. A prescription-based photobiomodulation system, comprising:
a device body comprising a hall effect sensor, a laser diode, a controller, a wireless communication module, and memory;
a plurality of interchangeable components, each interchangeable component comprising a different arrangement of magnets creating a distinct magnetic signature distinguishable by the hall effect sensor, wherein the different arrangement is defined by at least one characteristic selected from a quantity of magnets, relative positions of magnets, and/or polarities of magnets;
wherein the controller is configured to:
store associations between predefined magnetic signatures and corresponding operational parameters, the corresponding operational parameters comprising power level, treatment duration, and authorized usage count;
detect which interchangeable component from the plurality of interchangeable components is attached to the device body based on a magnetic signature sensed by the hall effect sensor;
automatically load a specific set of operational parameters from the memory based on the magnetic signature;
track a number of treatment cycles delivered with the interchangeable component;
disable device operation when the number of treatment cycles reaches the authorized usage count associated with the magnetic signature; and
receive wireless prescription data via the wireless communication module, the wireless prescription data comprising an updated authorized usage count, and update the authorized usage count in the memory to enable additional treatment cycles.

9. The system of claim 8, wherein the wireless prescription data further comprises component identification data specifying which interchangeable component from the plurality of interchangeable components is authorized for use, and wherein the controller is configured to verify that the magnetic signature matches an authorized magnetic signature specified in the component identification data before enabling device operation.

10. The system of claim 8, wherein each interchangeable component in the plurality of interchangeable components is associated with treatment of a different anatomical location, and wherein the controller is configured to load treatment procedures specific to an anatomical location based on the magnetic signature.

11. The system of claim 8, wherein the controller is configured to store treatment history data in the memory, the treatment history data comprising timestamps and power levels for each delivered treatment cycle, and wherein the wireless communication module is configured to transmit the treatment history data to a remote healthcare provider system.

12. The system of claim 8, wherein a first interchangeable component in the plurality of interchangeable components comprises two magnets creating a first magnetic field strength, wherein a second interchangeable component in the plurality of interchangeable components comprises four magnets creating a second magnetic field strength greater than the first magnetic field strength, and wherein the hall effect sensor is configured to distinguish between the first magnetic field strength and the second magnetic field strength.

13. The system of claim 8, wherein the wireless prescription data is received from a healthcare provider system after the healthcare provider system analyzes treatment outcome data transmitted from the device body via the wireless communication module.

14. The system of claim 8, wherein at least one interchangeable component in the plurality of interchangeable components is a battery component configured to supply electrical power to the device body and comprising a ring structure containing magnets arranged to create the distinct magnetic signature, and wherein at least one other interchangeable component in the plurality of interchangeable components is a disposable treatment tip comprising a fiber optic element and a tip body containing magnets arranged to create a different distinct magnetic signature.

15. A method of delivering prescribed photobiomodulation treatments comprising automatic component identification and prescription enforcement, the method comprising:
initializing a photobiomodulation device by powering on a device body comprising a hall effect sensor, a laser diode, a controller, and memory storing a procedure database, wherein the controller loads the procedure database into active memory;
detecting, by the hall effect sensor, presence of a magnetic field when an interchangeable component is brought into proximity with the device body, the interchangeable component containing a plurality of magnets;
measuring, by the hall effect sensor, magnetic field characteristics comprising field strength and spatial distribution of the magnetic field;
analyzing, by the controller, the magnetic field characteristics to determine a magnetic signature of the interchangeable component;
comparing, by the controller, the magnetic signature against a stored library of predefined magnetic signatures, wherein each predefined magnetic signature in the stored library is associated with a specific subset of treatment procedures, power level parameters, and authorized usage count;
automatically loading, by the controller, only the specific subset of treatment procedures corresponding to the magnetic signature from the procedure database, wherein treatment procedures not in the specific subset remain inaccessible;
restricting, by the controller, a user interface to display only the loaded specific subset of treatment procedures;
receiving user selection of a treatment procedure from the loaded specific subset of treatment procedures;
delivering a treatment cycle according to the selected treatment procedure by activating the laser diode;
incrementing, by the controller, a usage counter tracking a number of treatment cycles delivered;
determining, by the controller, that the usage counter has reached the authorized usage count;
capturing outcome data representing treatment results after completing the authorized usage count;
transmitting, via a wireless communication module, the outcome data to a healthcare provider system;
receiving, at the device body, a wireless authorization message from the healthcare provider system, the wireless authorization message comprising an updated authorized usage count; and
updating, by the controller, the authorized usage count in the memory based on the wireless authorization message to enable additional treatment cycles.

16. The method of claim 15, wherein the outcome data comprises photographic images of tissue treated during the additional treatment cycles, and further comprising:
analyzing, by an artificial intelligence system, the photographic images to detect changes in tissue characteristics selected from coloration, swelling, wound closure, and/or pigmentation;
generating, by the artificial intelligence system, a healing assessment report comprising quantified healing metrics; and
wherein the wireless authorization message is generated by the healthcare provider system based on the healing assessment report.

17. The method of claim 15, wherein the outcome data further comprises biomarker measurements selected from blood glucose level, oxygen saturation level, and/or temperature.

18. The method of claim 15, further comprising:
detecting, by the hall effect sensor, absence of the magnetic field when the interchangeable component is removed from the device body;
unloading, by the controller, the loaded specific subset of treatment procedures; and returning the device body to an initialization state wherein the device body is non-operational until a new interchangeable component is attached.

19. The method of claim 15, further comprising:
prior to detecting presence of the magnetic field, receiving wireless prescription data at the device body from the healthcare provider system, the wireless prescription data specifying an authorized magnetic signature;
after determining the magnetic signature, verifying, by the controller, that the determined magnetic signature matches the authorized magnetic signature; and
enabling device operation only when the determined magnetic signature matches the authorized magnetic signature.

20. The method of claim 15, wherein automatically loading only the specific subset of treatment procedures comprises:
identifying, by the controller, all treatment procedures in the procedure database associated with the magnetic signature;
making accessible only the identified treatment procedures through the user interface; and
preventing navigation to treatment procedures in the procedure database not associated with the magnetic signature.
